(19) United States
(12) Patent Application Publication
(10) Pub. No.: US 2004/0175377 A1
(43) Pub. Date: Sep. 9, 2004

(54) Agent and method for reducing intraocular pressure

(76) Inventors: Donna M. Peters, Middleton, WI (US); Paul L. Kaufman, Madison, WI (US)

Correspondence Address: Bennett J Berson, Quarles & Brady, PO Box 2113, Madison, WI, 53701-2113, US

(21) Appl. No.: 10/240,488

(22) PCT Filed: Mar. 29, 2001
(86) PCT No.: PCT/US01/10068

Related U.S. Application Data
(62) Division of application No. 60/192,942, filed on Mar. 29, 2000.

(51) Int. Cl. A61K 38/48; C12N 5/08

(52) U.S. Cl. 424/94.63; 435/368

(57) ABSTRACT

A method for reducing cell contacts and matrix organization in trabecular meshwork of a human or nonhuman eye includes the step of administering a suitable peptide having a sequence found in the Hep II domain of fibronectin where the peptide has an ability to disrupt cell contacts and matrix formation. A result of the disruption is reduced intraocular pressure in the treated eye.

### CROSS-REFERENCE TO RELATED APPLICATIONS

This application claims the benefit of provisional patent application No. 60/192,942 filed Mar. 29, 2000 which is incorporated herein by reference as if set forth in its entirety.

### STATEMENT REGARDING FEDERALLY SPONSORED RESEARCH OR DEVELOPMENT

Not applicable.

### BACKGROUND OF THE INVENTION

The present invention relates to identifying and using peptide agents for reducing intraocular pressure (IOP), more particularly to identifying and using peptide fragments of fibronectin, still more particularly to using the Heparin II (Hep II) binding domain of fibronectin and to identifying portions of the Hep II domain active in a method for reducing IOP.

Increased IOP is associated with a group of eye diseases called glaucomas, IOP in a normal eye is about 15 mm Hg; in glaucoma patients, the elevated IOP can range up to about 60 mm Hg. Clinically, glaucoma is characterized by progressive visual loss and blindness in its later stages due to the death of retinal ganglion cells (RGCs) which transmit visual signals from the eye to the brain. These cells are sensitive to pressure, and can be damaged or killed by elevated pressure in the eye. In some cases, the RGCs are so sensitive to pressure that even an IOP in the normal range is damaging Thus, all cases of glaucoma are treated by lowering IOP. In humans, IOP is determined, at least in part, by the resistance to aqueous humor outflow from the eye. The most common form of glaucoma is primary open angle glaucoma which is characterized by a malfunction of the trabecular meshwork (TM) which, in combination with the Schlemm's canal system, forms a specialized cellular tissue in the anterior chamber of the mammalian eye that regulates IOP. It is understood that the extracellular matrix ECM) in the tissue is also involved in maintaining normal aqueous outflow. The TM cells control the matrix biosynthesis and turnover rates. The extracellular matrix acts as a filter in the outflow channels that allows aqueous humor with cellular debris and particulate matter to pass. The trabecular outflow channels are kept free of obstructive debris by degradation and phagocytosis of matrix components. In glaucoma patients, the TM malfunctions, thereby obstructing the normal outflow of aqueous humor.

The extracellular matrix proteins are synthesized by and secreted from cells that line the TM. The matrix proteins include laminin, fibronectin, collagen (types I, III, IV, V, VI, and VIII), chondroitin, dermatan, and heparan sulfate proteoglycans, hyaluronic acid and a small amount of keratin sulfate. Fibronectin is a dimeric protein that includes three types of homologous, repeating sequences (Types I, II, and III repeats) clustered together to form discrete domains having distinct biological activities. The structure of fibronectin is disclosed in U.S. Pat. Nos. 4,839,464 and 5,019,646, the disclosures of which are incorporated by reference herein as if set forth in their entirety. Fibronectin interacts with transmembrane receptors that mediate signal transduction pathways. Among these receptors are members of the integrin family and a subfamily of heparan sulfate proteoglycans called syndecans. The TM is known to contain at least seven integrins including α3β1, αvβ1, α5β1, αvβ3, αvβ5, αvβ6, α4β1, and α4β7. The syndecans have not yet been identified in the TM. In normal processes, integrins and syndecans interact with the extracellular matrix proteins to build up a suitable ECM.

Pharmaceutical agents that can enhance aqueous outflow have been used therapeutically to reduce IOP. Since many cytoskeletal signaling pathways modulated by pharmaceutical agents are regulated by cell-matrix interactions, interactions between the cells and the ECM may also play an important role in modulating aqueous outflow. Known glaucoma treatments include miotic drugs that help open the eye's drainage system and facilitate fluid outflow by contracting the ciliary muscle within the eye (thereby mechanically expanding the TM), β-adrenergic antagonists, α₂-adrenergic agonists, and carbonic anhydrase inhibitors that decrease the rate at which fluid flows into the eye, prostaglandins that promote the outflow of aqueous humor through an alternate drainage route not involving the TM, and epinephrine which promotes fluid drainage through the TM. Each class of compounds has advantages and disadvantages, which are known to those skilled in the art. In addition to pharmaceutical therapies, surgical intervention is also a common glaucoma treatment. Glaucoma filtration surgery lowers IOP by creating a secondary outflow channel for egress of aqueous humor to the subconjunctival space. One shortcoming of glaucoma filtration surgery is that proliferating fibroblasts and fibrosis in subconjunctival space cause scarring that can lead again to an increase in IOP. Moreover, the pharmacologic agents used to overcome the negative effects of filtration surgery are themselves associated with further complications. Glaucoma filtration implants of the types described in U.S. Pat. No. 6,186,974 are also used to maintain open channels after glaucoma filtration surgery.

U.S. Pat. No. 6,013,628 (Skubitz et al.) disclose therapeutic methods employing peptides for treating diseases and conditions of the eye that include scarring and/or proliferation of fibroblasts. In particular, Skubitz teaches use of inter alia polypeptide FN-C/H-IV (amino acids 1784-1792 from the 33 kD fragment of the fibronectin A chain) for treating proliferative vitroretinopathy. Skubitz also teach methods for treating glaucoma, but only using a peptide containing isolated residues 49-60 from the NC1 domain of the type IV α2 collagen chain.

Various in vitro and in vivo animal model systems are used to study glaucoma and to evaluate glaucoma therapies. In particular, human eye organ cultures can be maintained for weeks during which the eyes in culture can be treated and compared against untreated eyes in culture. Therapies evaluated in human in vitro eye cultures are known to correlate well with effects observed in vivo. See, e.g., Erikson-Lamy, K, et al., Experimental Eye Research 50:143 (1990). For in vivo studies of ocular treatments, it is desirable to employ monkey model systems, which are well know in the art and are described, for example, in the following papers, incorporated herein by reference in their entirety: Gabelt, B. T. and Kaufman, P. L. Prostaglandin F₂ₐ increases uveoscleral outflow in the cynomolgus monkey. Exp Eye Res 1989;49:389-402; Gabelt, B. T. and Kaufman, P. L. The effect of prostaglandin F₂ₐ on trabecular outflow facility in cynomolgus monkeys. Exp Eye Res 1990;51:87-91; Gabelt, B. T., Crawford, K. and Kaufman, P. L. Outflow facility and its response to pilocarpine decline in aging rhesus monkeys. Arch Ophthalmol 1991;109:879-882; Tian, B., Gabelt, B. T., Peterson, J. A., Kiland, J. A. and Kaufman P. L. H-7 increases trabecular facility and facility after ciliary muscle disinsertion in monkeys. Invest Ophthalmol Vis Sci 1998;40:239-242; and Tian, B., Kaufman, P. L., Volberg, T., Gabelt, B. T.and Geiger, B. H-7 disrupts the actin cytoskeleton and increases outflow facility. Arch Ophthalmol 1998;116:633-643. The monkey model systems are preferred because the physiology of monkey eye drainage apparatus more closely resembles that of humans than does the drainage apparatus of lower mammals. For example, anterior chamber perfusions in the living cat and rabbit eye give more uncertain outflow facility values because of the tendency of aqueous humor to clot. It is also difficult to maintain stable anesthesia in a rabbit throughout a long experiment. Moreover, while eye organ cultures are very useful for these studies, other factors observed only in vivo, such as innervation, blood supply, and circulating hormones can influence how the therapeutic agent affects the TM and aqueous outflow.

More fundamental investigations of the physiology of the TM can be accomplished using known methods for culturing human TM (HTM) cells. HTM cell culture conditions are conventional and are described in the following papers, incorporated herein by reference in their entirety: Polansky J R. Wood I S. Maglio M T. Alvarado J A. Trabecular meshwork cell culture in glaucoma research: evaluation of biological activity and structural properties of human trabecular cells in vitro. Ophthalmology 1984; 91:580-95 and Alvarado J A. Wood I. Polansky J R. Human trabecular cells. II. Growth pattern and ultrastructural characteristics. Investigative Ophthalmology & Visual Science 1982; 23:464-78.

While various pharmacological and surgical methods for reducing intraocular pressue are known, all are associated with substantial risks and shortcomings. Accordingly additional methods are still sought by those skilled in the art.

### BRIEF SUMMARY OF THE INVENTION

The present invention is summarized in that a method for increasing outflow from a mammalian eye to reduce IOP includes the step of administering directly or indirectly to the TM of the eye an amount of a peptide sufficient to reduce IOP in the eye. In any embodiment, the peptide can be administered in combination with a covalently bound carrier or can be incorporated into a larger fusion protein which can contain tag sequences. The peptide can be administered directly or can be encoded by a polynucleotide sequence that encodes the peptide to be administered. Suitable modes of delivery are detailed below.

In a related aspect, the invention is summarized in that a peptide suitable for administration to achieve the goal of the invention is a Hep II domain of fibronectin that contains at least a portion of the type III 12-14 repeats.

In yet another aspect, a peptide suitable for administration in the methods of the invention comprises type III repeat 14, or repeats 13 and 14 or repeats 12, 13, and 14. At least a portion of a peptide active in the methods of the invention resides in repeat 14, although increased activity is observed when repeats 13 and/or 12 are administered in combination with repeat 14.

In a related embodiment, a peptide suitable for administration in the methods of the invention consists essentially of fibronectin type III repeats 12-14. In yet another embodiment, a peptide suitable for administration in the method consists of fibronectin type III repeats 12-14.

In still another embodiment of the invention, a peptide suitable for administration in the method includes amino acid sequence IDAPS and amino acid sequence PRARI, both of which are present in fibronectin type III repeat 14. The IDAPS sequence is known to bind the β subunit of integrin The PRARI sequence is known to bind syndecan and may bind to the α a subunit of integrin. Syndecan and integrin are extracellular matrix organizing proteins.

It is an object of the present invention to disrupt the TM and extracellular matrix of a mammalian eye to open natural channels for aqueous outflow to reduce IOP.

It is a feature of the present invention that the methods disrupt both cell contacts and matrix formation.

It is another feature of the invention that the methods employ peptides that can be administered locally and do not have the side effects associated with the pharmacological agents of prior methods.

It is an advantage of the present invention that it can avoid the need for surgical intervention to reduce IOP in glaucoma patients.

Other objects, features, and advantages of the invention will become apparent upon the consideration of the following detailed description taken in conjunction with the accompanying drawings.

### BRIEF DESCRIPTION OF THE SEVERAL VIEWS OF THE DRAWINGS

FIG. 1 is a schematic depiction of the fibronectin protein showing the type III repeats 12, 13, and 14 of the 33 kilodalton Hep II domain. The amino acid sequence of the entire fibronectin protein is known. The amino acid sequence of the Hep II domain is attached as SEQ ID NO:1.

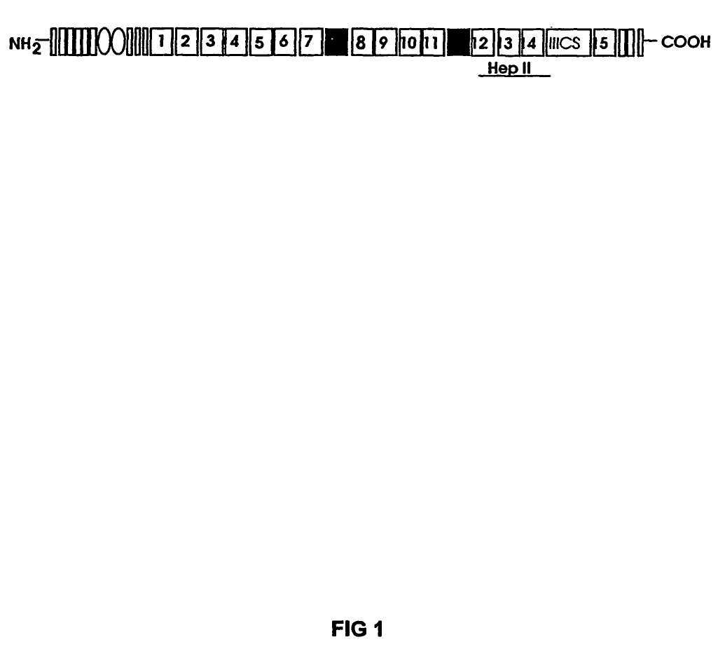

### DETAILED DESCRIPTION OF THE INVENTION

The inventors have identified the Hep II domain of fibronectin as having advantageous use in a method for reducing intraocular pressure (IOP) in a mammalian eye. In the method, a peptide within the scope of the invention is administered in a therapeutic amount to a human or non-human mammalian eye. An amount of a peptide is considered therapeutic if it reduces IOP, prevents an increase in IOP, or facilitates aqueous outflow by reducing resistance to outflow through the trabecular meshwork (TM).

In particular, the inventors have identified two pentapeptides in type III repeat 14 of the Hep II domain that reduce IOP when both are administered in a method of the invention. The Hep II domain is encoded by the polynucleotide set forth in SEQ ID NO:1 and having the amino acid sequence of SEQ ID NO:2. The pentapeptide amino acid sequences are IDAPS (amino acids 182-186 of SEQ ID NO:2) and PRARI (amino acids 205-209 of SEQ ID NO:2). The pentapeptides can be administered as separate peptides or as a single peptide or polypeptide molecule that includes both amino acid sequences. One such suitable peptide comprises the type III repeat 14 of fibronectin. The type III repeat 14 preferably has the amino acid sequence of that portion of human fibronectin, especially if the patient is a human. If the patient is a non-human animal, then the sequence of the type III repeat 14 is preferably that of the target animal species. In view of the substantial similarly in fibronectin proteins from all sources, the specific source of the protein is not considered critical to the invention.

In addition to the type III repeat 14 sequence, the administered molecule can also comprise repeat 13 of the Hep II domain or repeats 12, 13 and 14 of the Hep II domain, or can comprise a larger fragment of the fibronectin protein that includes at least one of the specified repeat portions of the Hep II domain. The peptides of the invention can be a free polypeptide or a polypeptide fragment coupled to a carrier molecule such as a biological carrier such as collagen, a glycosaminoglycan, a proteoglycan, a lipid, albumin or the like. The polypeptide can also be conjugated to a synthetic carrier such as a polymer, for example polyacrylic acid or polylactic acid. The carrier can also be an albumin or a cellulose molecule or a derivative of any of the foregoing.

In addition to being optionally linked a coupled carrier, the peptide can be administered in any conventional pharmaceutically acceptable delivery vehicle for administration. The particular means for delivering a peptide to the TM of the eye are not considered critical but rather are within the ability of a skilled artisan familiar with ocular physiology and fluid flow. Administration can be by drops, by intravitaeal or subconjunctival injection, by intraocular vitreal implantation of a slow release device such as that disclosed in U.S. Pat. No. 6,001,386 and patents cited therein. Drops are not a preferred delivery means because peptides are inefficiently delivered into the eye in this way. If an implant is employed it can include a synthetic or natural polymer or other suitable material such as methylcellulose, collagen, collagen sponges, hyaluronic acid/hyaluronate, sponges, a bioerodable polymer, a sustained-release membrane, and the like. Typically, an implant releases a therapeutic agent through diffusion or erosion of the implant with the resulting release of the therapeutic agent over a period of days or weeks. Intraocular injection of a suitable viral or non-viral vector carrying the gene encoding the peptide sequence, with expression primarily in the TM is also feasible. See, e.g., Liu, X., Brandt, C. R,. Gabelt, B. T. and Kaufman, P. L. Herpes Simplex virus mediated gene transfer to primate ocular tissues. Exp Eye Res 1999;69:385-395; Liu, X., Brandt, C. R., Gabelt, B. T. and Kaufman, P. L. Gene therapy for glaucoma: anterior and posterior segment targets, delivery systems, constraints. Ch 11. In: Gramer, E. & Grehn, F. Pathogenesis and Risk Factors of Glaucoma Berlin: Springer-Verlag, 1999:93-100; Kaufman, P. L. et al. A perspective of gene therapy in the glaucomas. Surv Ophthalmol 1999;43:S91-97; Borris, T., Masumoto, Y., Epstein, D. L. and Johnson, D. H. Gene transfer to the human trabecular meshwork by anterior segment perfusion. Invest Ophthalmol Vis Sci 1998;39:1503-1507; Borrás, T. et al. Trabecular meshwork gene transfer: its effects on intraocular pressure (IOP). Exp Eye Res 2000;71, Supplement 1:S3(Abs nr 8); Borrás, T., Gabelt, B. T., Rowlette, L. L. and Kaufman, P. L. Gene transfer to the trabecular meshwork of living monkeys can be followed non-invasively. Invest Ophthalmol Vis Sci 2000;41(ARVO Abstracts):S326(Abs nr 1718); Borrás, T., Rowlette, L. L., Erzurum, S. C. and Epstein, D. L. Adenoviral reporter gene transfer to the human trabecular meshwork does not alter aqueous humor outflow. Relevance for potential gene therapy of glaucoma Gene Ther 1999;6:515-524, all incorporated herein by reference as if set forth in their entirety.

In addition, genetic material encoding the peptide can be delivered to the eye by providing a genetic construct engineered to transcribe an encoded peptide or protein upon introduction into a host cell. The peptide- or protein-encoding portion of the genetic construct can be flanked on its 5′ end by a transcriptional promoter and on its 3′ end by a transcriptional terminator and can include any additional elements known to those of ordinary skill for enhancing or regulating transcription. Using the above-noted protocols, reporter gene expression has been observed for greater than 1 month in the TM of live monkeys injected into the anterior chamber with an adenovirus vector. A suitable vector is replication competent avirulent HSV (hrR3), although vectors that support longer term transgene expression may be available. It is also desirable to provide on the vector a promoter that supports high expression levels. Suitable promoters can include CMV-IE, EB4/5, ICP6, hER1-a, and hUb6. Of these the CMV is considered a preferred promoter.

A polynucleotide that encodes the peptide (or its structural or functional equivalent) can be determined by a skilled artisan with reference to the stated amino acid sequences or by reference to the known sequence that encodes the fibronectin protein. Bases 5702-5923 according to GenBank Accession Number X02761 are suitable. The skilled artisan will appreciate that certain changes to the nucleic acid or amino acid sequence will be functionally neutral and can be used interchangeably with the sequences described herein. It will also be understood that certain changes to the nucleic acid or amino acid sequence ray advantageously enhance or suppress the effect of the administered peptide.

The effective dose may vary with the extent to which outflow is blocked. The dose of the peptide administered will also depend upon the patient and the patient's medical history. The dose should be sufficient to reduce IOP and/or increase aqueous outflow. It is noted that the physics of the eye dictate that a greater pressure drop is observed upon treatment of a glaucomatous eye than upon treatment of a normal eye. Generally, the peptide can be administered in a solution containing the peptide at micromolar concentrations, say, on the order of 1-100 micromolar, for an extended time period, as required to reduce the blockage. Put another way, the peptide can be administered intracamerally in an amount between about 50 μg/ml and about 3 mg/ml, preferably between about 100 μg/ml and about 2 mg/ml, still more preferably between about 500 μg/ml and about 2 mg/ml, most preferably between about 750 μg/ml and about 1.25 mg/ml. Topical amounts would be at least about 100 fold higher. A practical constraint upon the amount of peptide administered is the amount at which the peptide itself begins to obstruct the tissue in the eye. At least 0.1% proteinaceous material in the administered material is known not to interfere with outflow function. Kee C, Gabelt B T, Gange S J, Kaufman P L, Serum effects on aqueous outflow during anterior chamber perfusion in monkeys. Invest Ophthalmol Vis Sci 37:1840-1848 (1996). Repeated administration may be indicated. A slow-release formulation is also suitable, as it is desired to maintain the peptide at an effective concentration after administration.

Without intending to be limited as to the theory of the invention, the inventors believe that integrin and syndecan cell surface receptors in trabecular meshwork (TM) cells are either saturated such that they are unable to bind to extracellular matrix (ECM) proteins or that the peptide provided acts as an agonist that disrupts other signaling events.

The invention will be further described by reference to the following examples which are intended to be merely exemplary and not limiting on the scope of the invention.

### EXAMPLE 1

The Hep II Domain Affects Organization of Cell Contacts and Extracellular Matrix

Differentiated human TM (HTM) cell cultures were incubated with a recombinant Hep II domain protein for eighteen hours. The effect on the extracellular matrix and cell contacts were observed using immunofluorescence microcopy, phase microscopy and western blot. Differentiated HTM cultures look like endothelial cells and form cell to cell junctions and closely resemble TM cells in vivo. Differentiated HTM cells also secrete matrix proteins such as fibronectin, type IV collagen and laminin. Non-differentiated cells lack these qualities and are less suitable for in vitro analysis methods. Cells were grown as described in Polansky J R, Weinreb R, Alvarado J A. Studies on human trabecular cells propagated in vitro. Vis Res 1981;21 :155-60 and Polansky J R, Weinreb R N, Baxter J D, Alvarado J. Human trabecular cells. I. Establishment in tissue culture and growth characteristics. Invest Ophthalmol Vis Sci 1979;18:1043-9, both incorporated by reference in their entirety.

After incubation, the turnover of fibronectin fibrils, a good indicator of matrix formation, was studied. In the presence of recombinant Hep II domain (at 4.6 μM), a dramatic decrease in the level of fibronectin matrixes was observed, suggesting that the added peptide triggered the removal of the matrix from HTM cell cultures. The Hep II protein was a GST protein. The GST portion was cleaved with thrombin as described by Bultmann, H., et al., Fibronectin Fibrilogenesis Involves the Heparin II Binding Domains of Fibronectin, J. Biol. Chem. 273:2601-2609 (1998).

It was also possible to replicate this observation by incubating the HTM cultures with monoclonal antibodies to the β1 or α4 subunits of α4β1 integrin, suggesting an involvement of α4β1 integrin-mediated signaling pathways. This observation was specific for these integrin subunits.

While this effect on matrix formation was relatively slow, the Hep II domain acts quickly in intact eyes to increase outflow facility. In human eye culture studies using pairs of control and test eyes, as described by Johnson, D H and Tschumper, R C. (1987) Human Trabecular Meshwork Organ Culture. IVOS 28:945-953, incorporated by reference, a baseline IOP was determined, pumps were turned off and the Hep II domain was injected at 13.8 μM into the anterior chamber of one eye. The pumps were kept off for four hours to allow the Hep II domain to mix with the contents of the anterior chamber. After four hours, the treated eyes were perfused with buffer containing the Hep II domain at 13.8 μM for twenty-four hours. Untreated eyes were perfused with buffer. When the pumps were turned on, pressure in the untreated eye returned to normal base line levels, while pressure in the treated eye showed a 32% decrease in IOP. After twenty-four hours, the pumps were turned off and the perfusate in both eyes was replaced with fresh buffer without the protein. In the absence of the Hep II domain, the pressure in the treated eye returned to the baseline level.

The treated and untreated eyes were then fixed and prepared for both light and electron microscopy. While the pairs of eyes were not distinguishable by visual examination using a light microscope, it was apparent from the electron microscopic analysis that the endothelium in Schlemm's canal was altered in the treated eye. Part of the inner side of the cannel was disrupted and small discontinuous cellular processes replaced a continuous cell layer found in a normal eye. This suggests a disruption in cell contacts resulting from the treatment The extracellular matrix was also altered and amorphous material appeared in the cribiform meshwork of the juxtacanalicular region, again suggesting changes to the matrix organization. These changes can account for the increased outflow in the presence of the Hep II domain since they would have created additional spaces or openings for fluid flow.

Further, a number of morphological changes also indicate disrupted cell contacts. These include an increase in the number of retraction fibers compared to untreated cultures and a retraction and rounding up of many cells at higher peptide concentrations. Finally, a significant increase in tyracine phosphorylation of vinculin was observed after treatment of the eye with the Hep II domain. Vinculin is a major structural protein that maintain cell-cell and cell-adhesion contracts. Changes in its phosphorylation state indicate that the treatment has biochemically affected the molecules that maintain cell-cell and cell-adhesion contracts. In summary, it appears that the Hep II domain can regulate outflow facility from the eye by at least two mechanisms which may or may not be mutually exclusive. The net result of the increase outflow facility is reduced IOP. These results have been verified in a plurality of assays in which six of seven treated organ-cultured eyes evidenced lower IOP after treatment with the Hep II domain.

### EXAMPLE 2

Location of Active Domain

To identify the active region of the Hep II domain, smaller fragments including either the Type III repeat 12, the Type III repeat 14, or the Type III repeats 13 and 14 were produced as recombinant GST proteins, as above. When the twelfth repeat was administered to HTM cell cultures as above, no activity was detected. The fourteenth repeat was active, but less active than peptides containing both the thirteenth and fourteenth repeats and still less active than the Hep II protein that contains the twelfth, thirteenth and fourteenth repeats. This suggests an active site in the fourteenth repeat with additional contributions to peptide confirmation from the twelfth and thirteenth repeats.

The inventors have also determined that the matrix blocking activity can be mimicked by antibodies to integrin α and β subunits. This is consistent with the presence in the fourteenth repeat of an integrin binding site having the amino acid sequence IDAPS and with the presence of a syndecan or integrin binding site having the sequence PRARI. When these pentapeptides were administered separately to HTM cell cultures, neither disrupted cell contacts or matrix assembly, suggesting that both sites play a role in reducing IOP.

The role of the PRARI site in the studied activity was further assessed by preparing a Hep II domain having a single point mutation in the pentapeptide. When the point mutation rendered the pentapeptide sequence PRARI, the ability to disrupt cell contacts and alter matrix organization was lost, further suggesting the importance of this pentapeptide in reducing IOP.

### Prophetic Example

In vivo Reduction of IOP

A peptide according to the invention is administered into at least one eye of a human or nonhuman patient having an IOP at least about 10 mm Hg higher than the average IOP in a human or non-human eye. The peptide is administered in an amount on the order of 4.6 μM for at least 3 hours. IOP is measured using conventional techniques both prior to and following treatment Reduced IOP is observed as long as treatment is continued and can be observed for a period of time thereafter.

The present invention is not intended to be limited to the foregoing, but to encompass all such modifications and variations as come within the scope of the following claims.

We claim:

1. A method for disrupting cell contacts and matrix formation in trabecular meshwork of a human or nonhuman eye, the method comprising the steps of:
administering to the trabecular meshwork an amount of a peptide sufficient to reduce cell contacts and matrix organization, the peptide comprising amino acid sequences IDAPS and PRARI.

2. A method for reducing intraocular pressure in a human or nonhuman eye, the method comprising the step of:
administering to the trabecular meshwork an amount of a peptide sufficient to reduce cell contacts and matrix organization, the peptide comprising amino acid sequences IDAPS and PRARI.

3. A method as claimed in claim 1 wherein the peptide comprises the fourteenth type III repeat of fibronectin.

4. A method as claimed in claim 1 wherein the peptide comprises the thirteenth and fourteenth type III repeat of fibronectin.

5. A method as claimed in claim 1 wherein the peptide comprises the twelfth, thirteenth and fourteenth type m repeats of fibronectin.

6. A method as claimed in claim 1 wherein the peptide comprises the Hep II domain of fibronectin.


SEQUENCE LISTING (2 sequences)

SEQ ID NO: 1 - DNA, 810 bp, Homo sapiens
  CDS (1)..(810)
attcctgcaccaactgacctgaagttcactcaggtcacacccacaagcctgagcgcccagtggacaccacccaatgttcagctcactggatatcgagtgcgggtgacccccaaggagaagaccggaccaatgaaagaaatcaaccttgctcctgacagctcatccgtggttgtatcaggacttatggtggccaccaaatatgaagtgagtgtctatgctcttaaggacactttgacaagcagaccagctcagggtgttgtcaccactctggagaatgtcagcccaccaagaagggctcgtgtgacagatgctactgagaccaccatcaccattagctggagaaccaagactgagacgatcactggcttccaagttgatgccgttccagccaatggccagactccaatccagagaaccatcaagccagatgtcagaagctacaccatcacaggtttacaaccaggcactgactacaagatctacctgtacaccttgaatgacaatgctcggagctcccctgtggtcatcgacgcctccactgccattgatgcaccatccaacctgcgtttcctggccaccacacccaattccttgctggtatcatggcagccgccacgtgccaggattaccggctacatcatcaagtatgagaagcctgggtctcctcccagagaagtggtccctcggccccgccctggtgtcacagaggctactattactggcctggaaccgggaaccgaatatacaatttatgtcattgccctgaagaataatcagaagagcgagcccctgattggaaggaaaaagaca

SEQ ID NO: 2 - protein, 270 aa, Homo sapiens
IPAPTDLKFTQVTPTSLSAQWTPPNVQLTGYRVRVTPKEKTGPMKEINLAPDSSSVVVSGLMVATKYEVSVYALKDTLTSRPAQGVVTTLENVSPPRRARVTDATETTITISWRTKTETITGFQVDAVPANGQTPIQRTIKPDVRSYTITGLQPGTDYKIYLYTLNDNARSSPVVIDASTAIDAPSNLRFLATTPNSLLVSWQPPRARITGYIIKYEKPGSPPREVVPRPRPGVTEATITGLEPGTEYTIYVIALKNNQKSEPLIGRKKT